(19) United States
(12) Patent Application Publication
(10) Pub. No.: US 2026/0272348 A1
(43) Pub. Date: Sep. 17, 2026

(54) Interstitial Fluid Sampling Device

(71) Applicant: SAMPLIMY MEDICAL AB, STOCKHOLM (SE)

(72) Inventors: Federico RIBET, Stockholm (SE); Niclas ROXHED, Bromma (SE); Göran STEMME, Lidingö (SE); Giorgio Massara, Varese (IT)

(73) Assignee: SAMPLIMY MEDICAL AB, STOCKHOLM (SE)

(21) Appl. No.: 19/673,114

(22) Filed: May 11, 2026

Related U.S. Application Data
(63) Continuation-in-part of application No. 17/794,671, filed on Jan. 22, 2021, now Pat. No. 12,622,615.

(30) Foreign Application Priority Data
Jan. 23, 2020 (SE) 2030019-0

(51) Int. Cl. A61B 5/15; A61B 5/00; A61B 5/151

(52) U.S. Cl. CPC A61B 5/150068; A61B 5/150022; A61B 5/150824; A61B 5/150984; A61B 5/15117; A61B 5/6831; A61B 5/6832; A61B 5/6843; A61B 2560/0223

(57) ABSTRACT

A device for extracting a fluid sample from a skin of a subject is disclosed. The device has at least one micro-needle having a tip portion, an end portion, and a passage. The tip portion is configured to be inserted into the subject's skin. The passage is configured to transport the fluid sample, extracted from the skin by the at least one micro-needle, away from the tip portion. The device also has a body that supports the at least one micro-needle and a storage unit arranged in the body. The storage unit is fluidically connected to the passage to collect and temporarily store the fluid sample transported by the passage. Additionally, a cutaneous force applicator applies a repeatable, controlled force to the skin tissue around the micro-needle to generate a controlled pressure on the skin tissue, to extract a fluid sample from the skin.

### TECHNICAL FIELD

The present disclosure generally relates to the field of sampling of biofluids, and in particular to techniques for extracting a fluid sample from the skin of a mammal subject, such as an interstitial fluid sample.

### BACKGROUND

To access information on the health of a patent beyond assessment of the symptoms, analyses of bodily samples need to be performed. Assessment of the concentration or presence of specific analytes in the body is an important aspect of medical practice for patient monitoring and disease diagnostics. Such an assessment allows for doctors and nurses to acquire information, either qualitative or quantitative, on the status of a patient and take medical decision based on thereon.

Depending on the disease or the condition to be assessed, different bodily matrices may be used, including tissue (for example via biopsy) and different fluids (both liquids and gases). Generally, it is common to use blood as a measurement matrix for a large variety of analytes, such as glucose, cholesterol, medical drugs, and the like. However, blood extraction is associated with invasive and painful methods, whether done via finger pricking or intravenously. Being able to monitor substances without the pain and discomfort associated with traditional blood sampling techniques such as venipuncture and finger pricks would therefore represent an improvement towards a more patient-centred healthcare. An emerging technology for accessing and sampling biofluids in a more convenient manner from the skin of subjects is micro-needle technology. In fact, micro-needle technology can be used, in various and different configurations, to extract small volumes of either blood or interstitial fluid (ISF) samples with minimal damage to the tissues. In particular, ISF is an emerging matrix for minimally invasive monitoring, which has shown a good correlation with blood values for several analytes (such as glucose) and can be extracted from within the skin.

ISF is a liquid present between tissue cells of the body, outside the cells themselves and the blood vessels. The composition of the ISF is known to be rather similar to that of blood plasma. In addition to water it may comprise salts, glucose, small proteins, lipids, amino acids, fatty acids, hormones, neurotransmitter, and other small biomolecules. The similarity is understood to be due to a molecular exchange between the blood in capillary vessels and the interstitial space occurring by diffusion, hydrostatic pressure, osmotic pressure, active transporters, and transcytosis across the capillary endothelium. Because of this similarity with blood plasma, ISF is gaining a growing interest as a monitoring matrix.

Examples of current methods for ISF sampling involve the use of hollow microneedles that are inserted intradermally, and wherein an ISF flow is induced into the needles by vacuum suction. However, such techniques are relatively complex to use and associated with several drawbacks relating to e.g. lack of portability due to the necessary pumping mechanism, incompatibility with current analytical standards in the clinics, sample storage and stability, and difficulties ensuring a well-controlled and repeatable sampling volume.

Alternatively, mechanical pressure into the tissue can be used as a mechanism to generate and drive the flow of fluid from within the skin to the sampling device. However, control and reproducibility of the applied pressure onto the skin tissue is crucial to the success of the sampling procedure, to ensure it exceeds the amount necessary to generate a flow, to complete the sampling procedure in a reasonable timeframe, and not to exceed a pressure that might end up detrimentally affecting the tissues and thus produced unwanted side effects.

Thus, there is a need for improved sampling techniques in general, and for an improved device for extracting ISF samples in particular.

### SUMMARY

It would be advantageous to achieve a technique overcoming or alleviating at least some of the above-mentioned drawbacks. In particular, it would be desirable to enable an improved device for extracting a fluid sample from the skin of a mammal subject, and do so in a controlled manner maximising the chances of success, while minimizing time and risk of side effects.

To better address one or more of these concerns, a sampling unit device and a holder having the features defined in the independent claims are provided. Preferable embodiments are defined in the dependent claims.

Hence, according to a first aspect, a device, also referred to as sampling unit in some examples, for extracting an ISF sample from the skin of a mammal subject is provided, comprising at least one micro-needle having a tip portion and a passage, wherein the tip portion is configured to be inserted into the skin of the mammal subject, and wherein the passage is configured to transport the fluid sample, extracted from the skin by the at least one micro-needle, away from the tip portion. The device further comprises a body configured to support the at least one micro-needle, and a storage unit, for example formed by a retaining material or liquid reservoir, arranged in a channel of the body. The retaining material is fluidically connected to the passage and configured to absorb and store the ISF sample transported by the passage.

According to a second aspect, a holder is provided, which is configured to receive a device according to the first aspect. The holder may also in some examples be referred to as an applicator, a casing, a pressure generating means, a cutaneous force applicator or an inserter. The holder comprises a rim portion configured to be placed on the skin and to at least partially encircle the at least one micro-needle when the at least one micro-needle is inserted in the skin. Further, the rim portion is configured to exert a pressure on portions of the skin adjacent to the at least one micro-needle to facilitate extraction of the fluid sample from the skin.

The above device and holder may further be used in a method in which the at least one micro-needle is inserted into the skin of the mammal subject and the ISF sample is extracted to the retaining material, in which the ISF sample is contained and stored. The extraction may be facilitated by the application of a pressure on portions of the skin adjacent to the at least one micro-needle, wherein the pressure may be applied by means of a rim portion of the holder.

The ISF or capillary blood sample, stored in the storage unit, may then be subject to biological or chemical analysis, e.g. performed by standard laboratory tools such as mass spectrometry. The chemical analysis may for example, but not limited to, be performed to detect drugs of abuse, such as phosphatidylethanol, amphetamine, MFM, MDMA, tetrahydrocannabinol (THC), cocaine and opiates. The analysis may be performed for quantification purposes or for merely indicating presence of these substances.

The micro-needle may be understood as a micro-scaled needle which due to its small dimensions can be inserted into the skin relatively painlessly and without causing any significant tissue damage, especially as compared to conventional hypodermic needles. The device may comprise a plurality of micro-needles, for example arranged in two-dimensional arrays. The micro-needle may be formed from a variety of materials such as silicon or glass, metals such as stainless steel, or polymers such as for example a hydrophilic polymer. Preferably, the micro-needle has a length that allows its tip to be arranged in the dermis of the skin of the mammal subject or create pores therein. Hence, it is understood that the dimensions of the micro-needle may vary depending on the sampling location, the actual thickness of the skin at the sampling location or the specific biofluid to be sampled. Various examples and dimensions of the micro-needle will be discussed later in connection with the description of different embodiments.

The micro-needle may hence be configured to penetrate the epidermis and provide access to the fluids in the dermal region, such as capillary blood or interstitial fluid. Due to its relatively small size, the use of the micro-needle may be relatively painless both during insertion and after removal, potentially improving patient acceptance and compliance. Further, by not drawing blood, in the case of ISF, and creating minimal wounds, in the case of both ISF and blood micro-sampling, the risks of infections, which may be significant especially in point-of-care applications, may be reduced. Moreover, the use of a micro-needle has been shown to reduce the risk for long term damage to the skin, which makes it an attractive technique for repeated sampling compared to collecting venous blood or finger pricking.

The micro-needle may comprise a passage configured to transport the extracted sample from the tip towards the storage unit. The passage may be a hollow interior of the micro-needle, also referred to as a lumen, or a trench being arranged in an outer surface of the micro-needle and extending along the length direction of the micro-needle. In some examples, the passage may comprise a hydrophilic surface portion to enhance capillary action and facilitate extraction and transport of the ISF or blood through the passage. In other examples, the micro-needle might be a solid micro-needle without a passage where upon retraction would leave a pore in the skin through which the fluid can exit the skin and then be transferred into a storage unit, such as a retaining material or reservoir, positioned outside the skin and attached as part of the sampling unit or the applicator.

A retaining material may be provided to absorb and hold at least some of the ISF sample extracted by the micro-needle delivered to the retaining material via the passage. This allows for the sampled fluid to be stored within the device, or at least within the retaining material, so that it can be shipped to a laboratory in a stable from and be analysed. Storing the ISF sample in the retaining material thus simplifies the logistics normally required by blood or plasma transportation, which may involve refrigeration and is thus costly. Additionally, biohazards may be reduced by handling the retaining material instead of the sample in its liquid form. Advantageously, the present aspects allow for the characterisation chain used in dried blood spots sampling techniques to be involved for logistics and analysis. This allows for a relatively simple logistics and measurement in small (few microlitres) sample volumes, which combined with the relatively painless extractions from the patient may allow the ISF to be used as monitoring matrix for a broader number of medical applications in the clinics and for point-of-care sampling.

Examples of the retaining material, which also may be referred to as a capillary means or a capillary storing means, may is include analytical grade paper, such as dry blood spot paper.

A pressure gradient may be employed to facilitate the extraction of the fluid sample from the skin. In the case of capillary blood, this could be important to start the bleeding process, since the wound created by a minimally invasive micro-needle or equivalent lancing mechanism is small and despite the internal bleeding caused by the incision of capillaries and the positive hydrostatic pressure, the skin might manage to elastically close and thus retain the bleeding despite the initially created opening, even after retraction of the micro-needle or lancing agent. Capillary blood may comprise capillary whole blood or capillary blood plasma. In the case of ISF, this is instead due to the fact that ISF tends to be retained by the skin and, unlike blood, does not flow out spontaneously as there generally is no net hydrostatically induced pressure present in the ISF. Thus, to facilitate extraction of the ISF sample an external force may be applied to collect the ISF sample from the skin. The external force may for example originate from a mechanical pressure, a vacuum suction, or capillary forces. A pressure generating means may therefore be employed to overcome the physiological ISF retention by the skin, start the extraction, and reduce the sampling time. The rim portion disclosed in connection with the second aspect is an example of such a means, which may be configured to exert a mechanical force on portions of the skin adjacent or close to the tip of the micro-needle when inserted in the skin. The mechanical force may induce an overpressure in the tissue, driving the ISF towards the micro-needle.

The device may be releasably attached to the pressure generating means, thereby allowing the device to be removed after sampling and the pressure generating means to be reused for subsequent samplings. Alternatively, the pressure generating means is integrated in the device.

The micro-needle may be arranged to protrude from the body, preferably orthogonally, such that the top portion of the micro-needle is allowed to penetrate the skin in response to the body being placed on the skin. Thus, the ISF sample may be collected by arranging the body on the skin and pushing the device towards the skin such that the micro-needle is forced into the skin of the patient. Preferably, the micro-needle may be arranged on an underside of the body that is relatively flat so as to provide mechanical support during the extraction of the ISF sample. The underside of the body, i.e., the surface of the body adapted to be placed on the skin during the sampling, may comprise an adhesive for providing additional mechanical support and reducing the risk for unintentional movement of the device during the sampling. Advantageously, the adhesive may assist in stabilising the position of the micro-needle when pressure is applied to adjacent portions of the skin.

The retaining material may be arranged to at least partly intersect a fluidic passageway formed by the passage of the micro-needle or by the pores left behind in the skin upon retraction of the micro-needle. In other words, the retaining material may protrude into a hollow interior of the micro-needle, or into a trench arranged in a side of the micro-needle, or be placed in proximity of the opened pores in the skin. In one example, the micro-needle may be provided with a groove or notch configured to allow the retaining material to directly contact the fluid transported in the passage. Further, the retaining material may be arranged to abut an end portion of the passage. Alternatively, or additionally, the passage may at least partly extend into the retaining material. Alternatively, the retaining material or reservoir may directly abut the flow, such as the expansion of a droplet being formed on the skin surface on top and surrounding the pore created by the micro-needle, on the outer side of the skin.

The above examples of the structural connection, or physical contact between the micro-needle and the retaining material, may advantageously serve the purpose of providing an efficient and reliable transport of the ISF fluid sample from the micro-needle into the retaining material, and reducing or minimizing the dead volume in the channel. In alternative examples there may however be a physical gap between the retaining material and the passage of the micro-needle, originating from the fact that the retaining material may be arranged at a distance from the passage of the micro-needle. The gap may for example be bridged by a portion of the channel in which the retaining material is arranged, allowing a fluidic connection by means of which the ISF sample can flow between the passage of the micro-needle and the retaining material even if there is no direct abutment between the retaining material and the passage of the micro-needle.

When the liquid front of the ISF sample has reached the retaining material, capillary forces of the retaining material may assist in the extraction of the remaining ISF sample from the skin and transport the ISF sample into the retaining material.

The retaining material may be understood as a material capable of absorbing and holding a liquid, such as ISF or capillary blood. The retaining material may be a porous material, which may be understood as a solid permeated by an interconnected network of pores or void. Preferably, the retaining material is hydrophilic so as to allow capillary filling and facilitate retention of the ISF sample. Examples of retaining materials include fibrous materials comprising cellulose (such as paper), glass fibres or polymeric fibres. The retaining material may be arranged to completely or at least partly fill the space forming the channel of the body. In one example, the retaining material may be provided as a sheet having a size that completely or at least partly covers a bottom surface of the channel.

The device may be provided with a fill level indicator to assist the user in determining when the predetermined amount or volume of ISF has been extracted and stored in the retaining material. This allows for the user to terminate the sampling at an appropriate time.

Several examples of fill level indicating mechanisms may be provided. In one example, a substance may be provided, configured to react with the ISF and change colour of the ISF sample. The substance may for example be provided in the retaining material, in which it may be mixed with the ISF as it progresses through the channel of the body. The change of colour may be into a colour that is relatively easy to detect for a user, for example through an inspection window arranged in a wall of the channel. This allows for the progress of the coloured ISF sample along the channel to be followed. The predetermined volume may for example be indicated by the coloured ISF sample reaching the window or a marking.

In another example, a metering capability, or fill level indication, may be provided by means of a functionalised line or region in the retaining material, configured to change colour upon wetting with the ISF.

Alternatively, or additionally, the fill level indicator may comprise a sensor or detector, which for example may be electrical or optical, configured to determine the fill level of the ISF sample in the retaining material. The sensor or detector may for example replace the optical inspection performed by the user as set out above. In an example, the fill level indication may be provided by means of an electrical resistance measurement configured to detect presence of ISF.

In a further example, time-control may be employed to determine or estimate the volume of the extracted ISF sample. Thus, the time during which the device is placed on the body and the tip of the needle inserted in the skin may be predetermined in order to control or at least affect the amount of ISF that is absorbed by the retention material.

The predetermined volume may in some examples be defined by the maximum volume the retention material is capable of receiving and storing. The maximum volume may in some examples be determined by the volume of the retention material. In case the retention material is a sheet-shaped material of a given thickness, the area of the sheet-sized material may be the factor defining the predetermined volume of the ISF sample. Thus, the volume metering of the sampled ISF may be determined by the geometrical dimensions of the retention material. Preferably, the predetermined volume may be 10 microlitres (μl) or less, such as for example 3 μl or less. In an example, the predetermined volume may be 1 μl or less. Such relatively small sample volumes (i.e. of a few microlitres) have the advantage that they may allow for a reduced sampling time.

The volume-metering, provided by the fill level mechanism and/or the predetermined filling volume of the retention material, advantageously allows for an improved quantitative analysis of the sample.

The tip portion of the micro-needle may have a radius of 50 micrometres (μm) or less, such as less than 5 μm or less. In an example, the radius is 1 μm or less. Further, the micro-needle may protrude 2000 μm or less from the body, such as 1000 μm or less. In an example, the micro-needle protrudes 500 μm or less. It will be appreciated that a reduced tip radius may be preferred in order to avoid or at least reduce the risk for damaging the tissue, and to reduce the risk for contaminating the ISF sample with blood. Further, it will be appreciated that the shape and in particular the length of the micro-needle may be selected to match the properties of the skin of the actual individual to be tested. Since the thickness of the skin, and the thickness and position of the dermis, may vary from individual to individual and also depend on the location on the subject's body, the micro-needle dimensions may be selected accordingly.

The passage of the micro-needle may be formed of a trench extending along at least a part of the micro-needle. In some examples, the passage may comprise a hydrophilic structure for facilitating transport of the ISF sample in the passage. The hydrophilic structure may for example comprise coating or layer increasing the hydrophilicity of the surface of the passage, and/or micro-pillars protruding from a surface of the passage. In a further example, the hydrophilic structure may comprise a porous material, preferably having a plurality of capillaries, absorbing the ISF sample and increasing the capillary action within the passage.

The pressure generating means, such as the holder, may comprise a resilient member configured to push the micro-needle towards the skin when the rim portion is placed on the skin. Advantageously, the resilient member is a spring, allowing the force by which the micro-needle is pushed against the skin to be determined by the spring constant of the resilient member rather than by the force by which the rim is pushed against the skin. Further, the pressure generating means may comprise an attachment means for securing the pressure generating means (and thus the micro-needle) to the skin and preferably push the rim of the pressure generating means against the skin to facilitate the extraction of the ISF sample. In case the locus is a forearm of the subject, the attachment means may be shaped as an armband.

According to a third aspect, a device for extracting a fluid sample from a skin of a subject is provided, comprising at least one micro-needle having a tip portion, wherein the tip portion is configured to be inserted into the skin of the mammal subject, and wherein the fluid sample is extracted from the skin through the pores in the skin generated by the at least one micro-needle. The device further comprises a body configured to support the at least one micro-needle, and a storage unit arranged and configured to contain and store the fluid sample.

According to a fourth aspect, a device for extracting a fluid sample from a skin of a subject is provided, comprising at least one micro-needle comprising a tip portion, an end portion, and a passage, wherein the tip portion is configured to be inserted into the skin of the subject, and wherein the passage is configured to transport the fluid sample, extracted from the skin by the at least one micro-needle, away from the tip portion; a body configured to support the at least one micro-needle; a storage unit arranged in the body, the storage unit fluidically connected to the passage and configured to collect and temporarily store the fluid sample transported by the passage; and a cutaneous force applicator configured to apply a repeatable, controlled force to the skin tissue around the micro-needle to generate a controlled pressure on the skin tissue, the pressure configured to extract a fluid sample from the skin.

The fluid sample extracted may be one of an interstitial fluid, or a capillary blood fluid.

The cutaneous force applicator may comprise a rim portion configured to abut the skin and to at least partially encircle the at least one micro-needle when said at least one micro-needle is inserted in the skin, wherein the rim portion may be further configured to exert a pressure on portions of the skin adjacent to the at least one micro-needle to facilitate extraction of the fluid sample from the skin.

The controlled pressure may be configured to provide the same amount of tissue compression and equivalent extraction efficacy, wherein extraction efficacy is defined by a unit of volume per unit of time, for example ul/sec.

The pressure applied may compress the skin tissue to induce transport of the fluid sample towards the pores generated in the skin by the micro-needle or, in different configurations, towards the tip portion and the micro-needle passage.

The pressure onto the skin may be applied manually by a human operator onto the device throughout the fluid extraction procedure. The cutaneous force applicator may be designed to provide a feedback to the human operator about the amount of pressure applied and/or about being in a specific range of the desired pressure. The cutaneous force applicator may further comprise a visual indicator having a movable mechanical part, wherein the visual indicator may be configured to provide the feedback about being in a specific range of the desired pressure, such as for example on a graded scale or color scale. The movable mechanical part may be connected to a spring mechanism being pulled or compressed with a force configured to apply the desired pressure to the skin tissue.

In certain configurations, after the device is initially set up, the controlled and reproducible pressure may be applied by the device to the skin tissue without further manual force from or control of the pressure by a human operator throughout the entire extraction of the fluid sample.

The device may further comprise an elastic band configured to secure the device to the skin and to apply a repeatable, reproducible, controlled pressure to the skin tissue via the rim portion of the cutaneous force applicator. The pressure may be measured by a pressure gauge on the elastic band wrapped around a location on the skin of the subject where the extraction procedure occurs. Optionally, the pressure gauge may be configured to expose a color change or a sign appearance in or through the elastic band due to a calibrated stretching of fibers and/or components of the elastic band.

When the pressure applied onto the device towards the rim portion surpasses a defined threshold, a mechanism may click and/or lock in position providing the feedback to the operator about readiness to start the extraction.

The cutaneous force applicator may be attached to the skin via an adhesive material. An amount of the pressure may be pre-defined by the indentation depth of the rim portion into the body tissue, which may be possible due to the elastic properties of the skin, with respect to the plane where the adhesive is attached to the skin, at least partially surrounding externally the rim portion.

The pressure may be applied on the skin via the rim portion by a spring-based mechanism, such as for example a metal coil spring, a curved plastic or steel leaf spring, a plastic bellow or flexural dome.

The cutaneous force applicator may further comprise a clip; the clip may comprise a spring-based mechanism arranged between two clip arms; the clip may be configured to pinch a portion of skin and tissue between the two clip arms.

The spring-based mechanism of the clip applies the desired force via the rim portion onto the skin tissue.

The applied force may be in a range between 0.5 N and 20 N; preferably the range may be between 4 N and 15 N; most preferably the range may be around 10 N±2 N.

The pressure, applied by the cutaneous force applicator to the skin surface, may be in a range between 0.05 MPa and 2.5 MPa (megapascals); preferably the pressure may be between 0.25 MPa and 1.5 MPa; most preferably the pressure may be around 1 MPa±0.4 MPa.

In a further example, in the device according to the previous embodiments, the sampling unit, comprising the body, the storage unit and at least one micro-needle, may not be monolithic with the rim portion of the cutaneous force applicator. In this case, the resulting force applied to the rim portion may be far greater than the force applied onto the sampling unit comprising the micro-needle. This weak coupling reduces the compression of the skin tissue around the micro-needle and, consequently, the resulting effective penetration depth, and thus reduces the risk of unwanted bleeding during ISF sampling procedures. Moreover, it can facilitate extraction by avoiding tissue compression where the fluid must flow out of the skin.

In a further example, in the device according to the previous embodiments, the rim portion may be geometrically configured to minimize the distance between its contact area with the skin and the orthogonal projection of the sampling unit on the skin; and the area of the rim may be designed to translate an applied force to a desired pressure into the body tissue. This configuration may help concentrate the pressure gradient in the skin tissue close to the extraction point (i.e. the micro-needle passage or generated pore), while minimizing its absolute value to avoid unwanted side effects or unnecessary subject discomfort.

Achieving extraction of fluid from the skin and ensuring that the extraction has worked are key parts of the success of a sampling procedure in a real setting. Therefore, an important aspect is to ensure and feedback to the user the success and completion of a procedure, for example by showing a color change in the retaining material or by any other mean of detection as described above. Moreover, for analytical purposes, it is of great importance to know the sampled volume and possibly guarantee the volume sampled is a known and always constant. This in fact enables quantitative sample analysis.

For this purpose, the sampling unit in any of the embodiments described above may be featured with a completion indication function, or additionally also a volume-metering function.

In an embodiment, the storage unit may contain a retaining material, such as a porous material, configured to allow removal of the retaining material from the storage unit for subsequent analysis. This storage unit may be made visible through a window within the applicator and/or the sampling unit enabling visualization of the progress of the colored fluid sample along the retaining material. In the case of ISF which is colorless, unlike blood, a coloring agent such as for example a dye may be used to facilitate visualization of the liquid front progressing within the retaining material. The same function may be realized in a liquid reservoir, such as a micro-fluidic channel. In a possible configuration of this embodiment, said retaining material or reservoir may have a pre-determined geometry that forces the fluid to fill it in a deterministic manner so that a specific portion of the retaining material is always filled last during a sampling procedure. Therefore, when the geometry is filled with fluid, and the geometry corresponds to the specific volume to be sampled, the operator may be able to recognize a complete sampling procedure, having collected a pre-determined volume of fluid, thus enabling subsequent quantitative analysis of the sample. Taking state-of-the-art alternative examples as comparisons, a typical dry blood spot (DBS) spreads irregularly on a paper disc, not filling the paper, and being absorbed and spreading in a non-deterministic way in it, preventing control of its volume and not enabling a filling completion indication.

Additionally, it is important to note that in this embodiment the best geometry might not be an obvious straight path, such as that of a lateral flow assay or a simple micro-channel. In fact, it may be the result of a trade-off, optimizing its fit into the sampling unit, usability during analysis, as well as time-resolution to stop the procedure. On one hand, given a certain volume and two-dimensional area of the retaining material needed to collect said pre-determined fluid volume, the geometry should minimize the orthogonal projection of the body on the skin surface to facilitate efficient sampling by minimizing the distance between the rim and the micro-needle, as well to enhance fitting in the subsequent analytical process, such as fitting, preferably flat on the bottom, into the receiving wells of a 96-well-plate or similar. On the other hand, to minimize fluidic resistance as well as provide enough time resolution during the filling procedure for the operator to visualize and stop the process timely. For this reason, a different geometry such as the one illustrated in the drawings may be necessary to optimize this trade-off between volume-metering precision, sampling speed, sampling efficacy and usability for subsequent analysis.

Other objects, features and advantages of the enclosed embodiments will be apparent from the following detailed description, from the attached dependent claims as well as from the drawings. Those skilled in the art realise that different features of the present invention, even if recited in different claims, can be combined in embodiments other than those described above and in the following. For example, the embodiments disclosed in connection to the fourth aspect also apply with respect to the third aspect. For example, the holder according to the second or fourth aspect may form part of the device according to the first aspect. Similarly, the rim portion of the holder according to the second or fourth aspect, configured for exerting a driving overpressure guiding the fluid flow into the micro-needle, may form part of the device according to the first aspect. Further, it should be noted that the present disclosure is not necessarily limited to extraction of ISF or capillary blood samples from human patients. The device disclosed herein may also be used for ISF or capillary blood sampling for veterinary purposes, even though it is appreciated that the dimensions of the micro-needle may be adjusted to suit the skin of the actual individual or species in question.

### BRIEF DESCRIPTION OF DRAWINGS

Exemplifying embodiments will now be described in more detail with reference to the following appended drawings, on which:

FIG. 1 is a cross section of a device according to an embodiment, when placed on the skin of the mammal subject;

FIG. 2 is a cross section of a device when attached to a holder according to an embodiment;

FIG. 3 is a perspective view of an array of micro-needles having a trench and a hydrophilic structure according to an embodiment;

FIG. 4a is a top-view illustration of the sampling unit in one possible embodiment, composed of one micro-needle placed in the center of a circular body, perpendicularly to the view plane, and a storage unit containing a porous retaining material to temporarily store and preserve the sample;

FIG. 4b is a cross section of a device comprising a fill level indicator according to another embodiment;

FIG. 4c is a side cross-sectional view of an embodiment of the device comprising a sampling unit and a cutaneous force applicator unit;

FIG. 5a is a perspective view of the device when placed on a forearm of a subject according to an embodiment;

FIG. 5b is a perspective view of the device when placed on a forearm of a subject according to another embodiment;

FIG. 6a is a side view of the device when it is in the rest position according to an embodiment;

FIG. 6b is a side view of the device when a force applied from the upper side is therefore moving the pressure indicator vertically according to an embodiment;

FIG. 7a is a cross section of the device when it is in the rest position according to an embodiment;

FIG. 7b is a cross section of the device when a force applied from the upper side is therefore moving the pressure indicator vertically according to an embodiment;

FIG. 8a is a perspective view of the applicator comprising an elastic band to secure the applicator to the body and apply a force onto the rim when placed for example on a forearm of a subject according to an embodiment; where thus the attachment means is an elastic band exercising a specific force/pressure amount, via the rim onto the skin tissue, defined by the elasticity and locking in place of the elastic band;

FIG. 8b is a perspective view of the applicator comprising an elastic band to secure the device to the body and apply a force onto the rim when placed on a forearm of a subject according to an embodiment, where the elastic band comprises a force gauge able to provide information about its stretch and thus, in turn, on the force applied to the rim applying pressure on the skin tissue around the sampling unit; in this embodiment the force gauge is implemented by exposing a color change or revealing a sign in or through the elastic band due to the calibrated stretching of the fibers/components of the elastic band itself, when stretched beyond a certain threshold force;

FIG. 8c is a perspective view of the device comprising an elastic band to secure the device to the body and apply a force onto the rim when placed on a forearm of a subject according to an embodiment; in this embodiment the force gauge is implemented by visualizing a scale, color-coded or graded, though a slit or opening within the elastic band, which gets more exposed the more the elastic band is elongated, allowing the operator to tune and fix the amount of force applied. In a similar alternative embodiment, a similar visualization concept might occur on the side of the elastic band and result in the alignment of certain signs on the elastic band with the graded or color-coded scale upon stretching with a certain force;

FIG. 9a is a perspective view of the device when placed on a forearm of a subject, where the applicator is attached to the skin via an adhesive material according to an embodiment;

FIG. 9b is a top view of the device support where the device is attached to the skin via an adhesive material according to an embodiment;

FIG. 10a is a perspective view of the cross-section of the applicator where the central cylindrical component is rigid and its bottom is acting as the rim providing force/pressure onto the skin according to an embodiment;

FIG. 10b is a side view of the same applicator;

FIG. 11 is a side view of the device where the force/pressure is applied on the skin via the rim by a spring-loaded mechanism, such as for example a metal coil spring, a curved plastic or steel leaf spring, a plastic bellow or flexural dome according to an embodiment;

FIG. 12a is a perspective view of the applicator where the mean to attach to the body is a clothespin-like or clip-like mechanism used to pinch a portion of skin and underlying tissue, and where the spring-based mechanism of the clip itself applies the desired force via the rim onto the skin tissue according to an embodiment;

FIG. 12b is a side view of the same applicator in the closed position, where between the two clip plates a skin portion would be pinched according to an embodiment; and

FIG. 12c is a side view of the same applicator in the open position, where between the plates a skin portion is present. Upon closing the clip mechanism, the skin is pinched in position and the pressure is applied to the skin tissue via the rim portion on the bottom side of the top plate of the clip.

FIG. 13 is a perspective view of an embodiment where the micro-needle may be retracted and the sample directly collected into a storage unit, such as a retaining material, when exiting the pores left in the skin, without flowing within the passage of a micro-needle.

As illustrated in the figures, the sizes of the elements and regions may be exaggerated for illustrative purposes and, thus, are provided to illustrate the general structure of the embodiments. Like reference numerals refer to like elements throughout.

### DETAILED DESCRIPTION

Exemplifying embodiments will now be described more fully hereinafter with reference to the accompanying drawings, in which currently preferred embodiments are shown. The invention may, however, be embodied in many different forms and should not be construed as limited to the embodiments set forth herein; rather, these embodiments are provided for thoroughness and completeness, and to fully convey the scope of and motivation behind the invention to the skilled person.

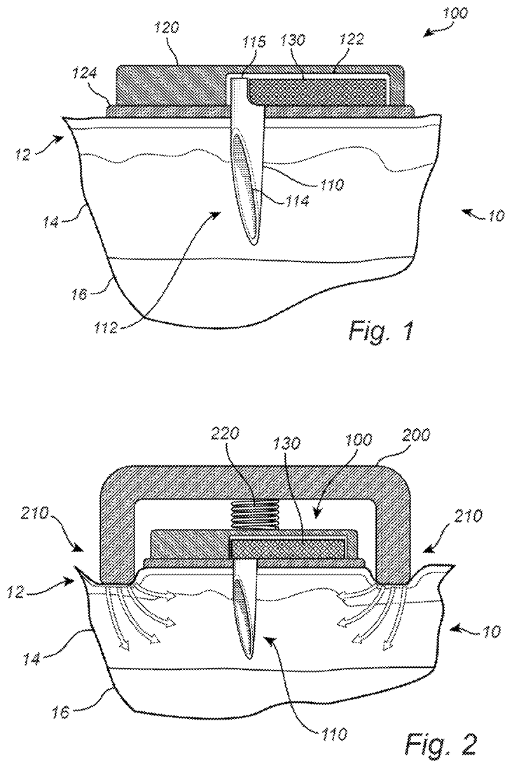

FIG. 1 is a cross section of a sampling unit device 100 according to an embodiment, comprising at least one micro-needle 110 comprising a tip portion 112 and a passage 114. The micro-needle 110 is supported by a body 120, comprising a retaining material 130 arranged in a channel 122 of the body 120. The retaining material 130 may be fluidically connected to the passage 114 and configured to absorb and store an interstitial fluid sample extracted from the skin 10 of a mammal subject. As indicated in the figure, the tip portion 112 is configured to be inserted into the skin 10 of the mammal subject, wherein the passage 114 is configured to transport the ISF sample, which is extracted by the micro-needle 110, away from the tip portion 112 and into the retaining material 130. Further, an adhesive (not shown) may be provided on an underside of the device 100, between the body 120 and the skin 10, for temporarily attaching or securing the device 100 to the skin 10 during the sampling. The adhesive may for example be a medical grade adhesive, preferably applied in a relatively thin layer of about 50 μm.

Generally, there are two main layers of the skin 10: epidermis 12 and dermis 14. Below dermis 14, a third layer of tissue is present; the hypodermis 16 (or subcutaneous tissue). The outermost layer, the epidermis 12, serves as a waterproof barrier enclosing the body of the subject and acts as a protection against infections. The middle layer, the dermis 14, protects the body from external stress and strain, and hosts thermo-and mechanoreceptors. The subcutaneous tissue 16 mainly consists of connective and fat tissue. Its main purposes are to attach the skin to muscles and bones, and to connect nerves and blood vessels to the skin 10. The thickness of the different layers strongly varies across different body locations and between different species, with the overall skin thickness of the human ranging from 0.05 millimetres on the eyelids to more than 1.5 millimetres on the feet soles. Considering the human forearm, the typical location used for example for traditional blood sampling via venipuncture, the average skin thickness is about 1 millimetre.

ISF is a liquid present in all parts of the body between tissue cells, outside the cells themselves and the blood vessels. Because its similarity with blood plasma, ISF is gaining a growing interest as a monitoring matrix. In particular, skin ISF is very interesting for monitoring applications because of its accessibility. Moreover, the concentration of several bioanalytes is closely correlated to the one in blood, which is typically used as a medical standard. Substances such as glucose, for example, are present in approximately the same concentration as in blood, with just a short physiological delay, quantified in the order of 4 to 12 minutes, due to the diffusion time from the blood capillaries. Additionally, by targeting skin ISF, monitoring is not only potentially achieved in a minimally-invasive fashion but continues monitoring with transdermal or intradermal devices can also be performed, providing a complete temporal picture of the evolution of the targeted analyte concentration.

Among the different skin compartments, the dermis 14 has shown to proportionally contain the largest amount and the most homogenously distributed ISF. Thus, the device 100 is preferably configured to allow the tip portion 112 of the micro-needle 110 to be arranged in the dermis 14 when the body 120 is placed on the skin 10 as illustrated in the present figure. However, the device 100 may also be configured to extract ISF from other parts of the skin 10, such as for example the hypodermis 16. The micro-needle 110 may for example protrude about 1 mm from the body 120 of the device 100, which has shown to be suitable for arranging the tip portion 112 in the dermis 14 of the forearm of a human. Further, the tip portion 112 may have a radius of curvature of about 5 μm to facilitate penetration of the skin. The micro-needle 110 according to the present example may be formed of stainless steel, which may reduce the cost compared to for example silicon-based microneedles and allow for standard manufacturing processes for needle cutting and shaping to be used. In the present example the micro-needle 110 is formed from a 32 G (Gauge) stainless steel needle processed by means of a femtosecond laser and cleaned in a 10% citric acid solution in DI water and sonicated, resulting in hydrophilic surfaces.

Capillary whole blood, or plasma, requires instead not only access to the tissue within or underneath the skin, but also to sever at least one capillary to start a bleeding in the process. Therefore, the damage created is typically larger than that of an ISF sampling procedure, the object is to avoid bleeding. To create a bleeding from the skin capillaries, a needle or blade integrated into a lancing mechanism is typically used, such as those of finger-prick lancets. Commonly, needles and blades are also made of metals such as stainless steel, or more rarely of hard polymers or silicon.

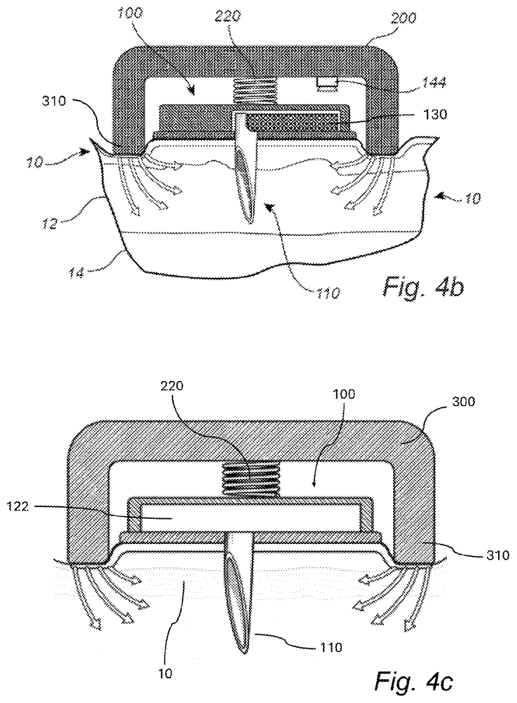

The end portion 115 opposite to the tip portion 112 may be arranged within the body 120, preferably in contact with the retaining material 130. The retaining material 130 may for example be arranged to overlap with a meniscus of fluid, such as ISF or blood, protruding from the end portion 115 of the micro-needle or be arranged to intersect the flow passageway formed by the lumen 114 of the micro-needle 110. In a further example, shown in for example FIG. 2 below, the end portion 115 of the micro-needle 110 may be interfaced to the retaining material 130 such that the opening of the lumen 114 is arranged to line up with a surface, edge or corner of the retaining material. In a further example, as later illustrated in FIG. 4c, the micro-needle passage may be simply interfaced with a liquid reservoir, in the form of a fluidic channel or chamber. In the present example, as illustrated in FIG. 1, the end portion 115 of the micro-needle comprises a notch in which a portion of the retaining material 130 can be received to at least partly intersect the flow passageway formed by the lumen 114, thereby bridging the fluid flow. In the present example, the total length of the micro-needle is about 1.9 mm and the portion protruding from the body 120, preferably orthogonally from the lower surface of the body 120, about 1 millimetre.

The length and geometry of the needles, or of any pricking element, may be adjusted depending on body location, skin type and condition, and on the fluid to be targeted for extraction.

The body 120, in which the micro-needle 110 and the retaining material 130 may be arranged, may be formed by for example a laminate of two or more layers. The body 120 may in an example comprise a base 124 of PMMA, on top of which two 170 μm thick layers of a double-adhesive tape and a plastic foil may be arranged, forming the channel 122 accommodating the retaining material 130. In other examples the body may be formed from milling, silicon processing, moulding or 3D printing.

The retaining material 130 may be arranged to at least partly fill the channel 122 of the body 120. In the present example, the retaining material 130 is formed of a sheet of grade 238 paper that is processed to obtain a geometry able to store about 1 μl of liquid. An example of such a geometry will be discussed in connection with FIG. 4a.

During sampling, the base 124 of the body 120 may be arranged on the skin 10 of the mammal subject such that the tip portion 112 of the micro-needle penetrates the epidermis 12 and proceeds into the dermis 14. ISF of the skin 10 may then flow into the passage 114 of the micro-needle 110 and further into the retaining material 130 in the channel of the body 120, where the ISF sample may be stored. After the sampling, the device 100 can be removed from the skin 10 and stored for subsequent analysis.

The flow of ISF into the micro-needle may be facilitated or driven by capillary action of the passage 114 of the micro-needle and/or the retaining material 130, or by means of a pressure gradient generated by an applied mechanical pressure or a vacuum suction. An example of a technique employing an applied mechanical pressure will be discussed in the following with reference to the embodiment of FIG. 2.

FIG. 2 schematically illustrates the application of a mechanical pressure to the skin adjacent the micro-needle to facilitate extraction of the ISF sample. The mechanical pressure may be generated by a pressure generating means 200 configured to be placed on the skin 10 to exert a pressure on portions of the skin to drive ISF into the micro-needle 110, or more generally toward the pore generated by the pricking of the micro-needle into the skin. The pressure generating means 200 may for example comprise or be formed of a housing, or holder, having one or several rim portions 210 adapted to be pushed against the skin 10 to generate a local, hydrostatic overpressure (indicated by arrows in the figure) in the ISF of the skin 10 during the sampling. While it is appreciated that the pressure generating means 200 may be structurally integrated in the device 100 as described above with reference to FIG. 1, in another example h the pressure generating means 200 is a reusable inserter or holder and the device 100 is a disposable sampling unit which can be attached and detached from the inserter.

The pressure generating means 200 according to the present example may comprise a rim portion 210, which for example may be ring-shaped and at least partly encircle the micro-needle when placed on the skin 10. This allows for a pressure to be generated, which may converge radially towards the micro-needle to overcome the physiological ISF retention exercised by the skin or the closure of the generated pores. The rim portion 210 may form the open end of a casing or housing 200 configured to receive the sampling device 100 of FIG. 1. The sampling device 100 may be movable relative to the casing 200, allowing the rim portion 210 to exert a first force on the skin 10 and the sampling device 100 to exert second force on the skin, different from the first force. A resilient member, such as a spring element 220, may be arranged to keep the device 100 in the desired position in relation to the pressure generating means 200, or casing 200, and to allow for the micro-needle 110 to penetrate the skin 10. Further, the spring element 220 may be employed to push the sampling device 100 against the skin 10 during the sampling and thereby assist in keeping the device 100 in the desired position. The resilient member 220 may for example extend between an inner wall of the casing and a top surface of the sampling device 100. The resilient member 220 may for example be formed as a helical spring or a flat spring, or any other flexible structures capable of resiliently guiding the sampling device 100 in a direction towards the skin 10 as the casing 200 is placed onto the skin 10.

It should be noted that the sampling device 100 may be similarly configured as the device 100 discussed in connection with FIG. 1. However, in the present example, the sampling device 100 differs from the device 100 of FIG. 1 in that the end portion 115 of the micro-needle 110 is interfaced into the retaining material 130 without an enhanced overlap area of the liquid front meniscus progressing from the passage 114 towards the retaining material 130.

In further embodiments, however, a system is envisioned comprising three separate functional components: the device 100 as described above, a pressure generating means comprising a rim portion 210 as illustrated in for example FIG. 2, and an inserter for receiving and positioning the device 100. The inserter may be spring-loaded, comprising a resilient member 220 assisting in the positioning of the device 100 and, possibly, push the device 100 towards the skin 10 during the sampling. Preferably, the inserter is adapted to be reusable. The pressure generating means may in some examples be attached to the inserter. Further, the pressure generating means may be fixed to the inserter or moveable relative to the inserter.

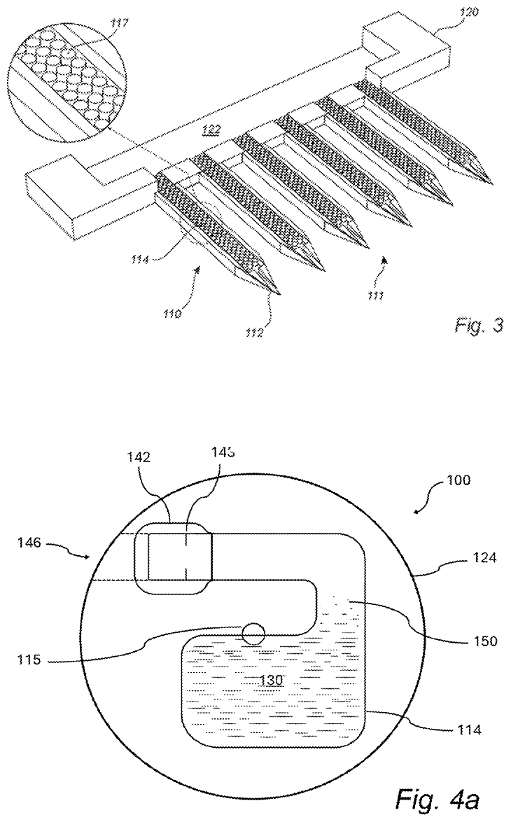

FIG. 3 shows a portion of a device 100 which may be similarly configured as the devices 100 discussed in connection with FIGS. 1 and 2. The device of in FIG. 3 however differs in that it comprises more than one micro-needle, in the present example an array 111 of 6 micro-needles 110, formed from a semiconductor material such as for example silicon, using conventional semiconductor processing methods. The array 111 of micro-needles 110 may for example be arranged as an in-plane array 111 of the silicon substrate, with the micro-needles 114 extending in the main plane of extension of the silicon wafer substrate.

As indicated in the figure, the micro-needles 110 may be side-opened, i.e., having a passage 114 formed as a trench extending along a side of the micro-needle 110. The trench 114 may extend from the tip portion 112 of the micro-needle to the end portion 115 and fall into a channel 122 which may be common to all the micro-needles 110.

The channel 112 may comprise a structure 117 for increasing its hydrophilicity. In the present example, such a hydrophilic structure may be formed by a plurality of micropillars 117 protruding from an inner surface of the channel 112. The micropillars 117 may for example be formed when etching the channel 112 and may hence be arranged to protrude from a bottom surface of the channel (i.e., the surface opposing the open wall).

The device 100 may comprise one or several micro-needle arrays and is not limited to the array 111 and number of micro-needles 110 depicted in the present figure.

FIG. 4a shows a cross-sectional top view of a device 100 according to an example, which may be similarly configured as any of the devices 100 shown in FIGS. 1-3 and FIG. 4c. The body 124 comprises a channel 114 that extends from the end portion 115 of the micro-needle to a fill level indicator 143, in the present example comprising a visualization window 142. Further, the body 124 may comprise a vent hole 146 connected to the end of the channel for allowing air to escape the channel 114 as the liquid front 150 of the fluid sample progresses through the channel 114. The geometries of the channel 114, the window 142 and the vent hole 146 may be for example formed by laser cutting, using a carbon-dioxide laser, or by means of milling, 3D printing, cutter plotting, or moulding.

The retaining material 130, such as for example a paper configured to absorb ISF or blood, may be arranged in the channel 114 and provided with a shape that conforms with the channel 114. Thus, the amount of fluid that can be held by the paper, i.e., the maximum volume of the fluid sample, may be determined by the shape and size of the retaining material 130, also in case the channel 114 should have a shape differing from the shape of the retaining material 130. During operation, the fluid sample may be extracted by the micro-needle 110, 115 and transported into the channel 114, where it is absorbed by the retention material 130 or stored directly into a reservoir in liquid form. The progress of the absorption of the fluid sample may be indicated by the liquid front 150 spreading in the channel 114 and moving towards the vent hole 146 and the window 142, where it can be detected either manually (visually) by a user or by means of a sensor or detector as illustrated in FIG. 4b. In one example, the fill level indicator 143, or any part of the retaining material 130 that is crossed by the liquid front 150 moving towards the window 146, may comprise a substance configured to change a colour of the ISF sample. The substance may for example be a dry blue dye powder that has been added in a few micrograms to the retaining material 130 to be mixed with the ISF and to enable visualization of the front of the ISF flow during sampling.

The fill level indicator 143indicates a predetermined fill level, allowing for the user to stop the sampling by removing the device 100 from the skin 10 when the liquid front 150 has reached the marking 143. The marking 143 may for example be arranged such that it is visible through the window 142.

The geometry for the retaining material, such as a paper strip 130, may together with the fill level indicator 143 be used to ensure that the correct amount of fluid is absorbed before the sampling is stopped. Thus, by verifying that the liquid front 150 has reached the window 142 or marking 143 before the device 100 is removed from the skin 10, it can be assumed that a sufficient amount of fluid is present in the retaining material 130. To further improve quantification, a portion of the retaining material 130 can be used for the subsequent analysis. The portion may for example be selected to include the region closest to the micro-needle to ensure that at least that portion is completely filled with fluid. By selecting the size of the portion with high accuracy, a correspondingly high accuracy of the sample volume may be obtained.

Additionally, in this current example, a specific geometry of the retaining material is illustrated. The purpose as described above, is to ensure the retaining material 130 at the proximal end, in the proximity of the micro-needle 110,115, is always completely filled with fluid before the fluid front 150 can reach the visualization window 142 at the distal end, while maximising the usage of area within the body and thus minimize the diameter of the body 124 necessary to collect a specific amount of fluid.

FIG. 4b shows a side view of the device of FIG. 4a when inserted in a holder 200 according to an example. The holder 200 may be similar to the one discussed above in connection with FIG. 2, which the addition of a sensor or detector 144 arranged to observe the fill level of the ISF sample in the retaining material 130. The sensor or detector 144 may for example be of an optical type or of an electrical resistance type, configured to monitor the window 142 and to output a signal carrying information indicating that the liquid front 150 has reached the window 142 or the marking 143.

A device is typically composed of a sampling unit and a pressure generating means or cutaneous force applicator. FIG. 4c shows a side cross-sectional view of an embodiment of the device comprising a sampling unit and a cutaneous force applicator. The sampling unit comprises one or more micro-needles, responsible to for penetrating skin and providing access to the fluids contained therein, a support material, and a reservoir or retaining material responsible for collecting the extracted fluid. The cutaneous force applicator, in the various embodiments, serves two main purposes: first, it is responsible for the application of the sampling unit to the skin and achieving controlled penetration of the at least one micro-needle into the skin, most typically via a spring-loaded mechanism, and second, it is responsible to apply a certain force towards the skin tissue 10, resulting in an applied pressure to the skin surface, transferred into the tissue and thus generating a positive pressure in the tissue guiding fluid towards and into the micro-needle passage, according to the different embodiments. The former application function is overly simplified in this illustration by a coil spring 220. Typically, the application function is activated via a button or similar actuation mechanism, not shown in FIG. 4c.

The pressure is typically applied by a rim 320, at least partially surrounding the sampling unit. To achieve the desired technical effect, i.e. apply sufficient but not excessive pressure to the skin tissue so that such positive pressure guides the fluid contained therein towards the micro-needle or the pores resulting from the micro-needle penetration and then into the passage towards the storage unit within the body of the sampling unit.

It is of great importance for the success of the sampling procedure that the applied force and resulting pressure are controlled and within a certain range. Below a certain pressure provided to the tissue, no fluid might be extracted due to the liquid retaining capacity of the skin tissue itself, while an insufficient pressure might result in too slow extraction to serve the clinical scope envisioned. On the opposite side, an excessive force/pressure can result in discomfort for the subject, as well as unwanted side effects which may include bleeding and bruising.

Moreover, it is important to apply the pressure in a close proximity to the sampling unit to avoid dispersion of the pressure far away from the collection point, consisting of the passage of the at least one micro-needle present in the sampling unit. In fact, the technical effect is obtained by compressing the skin tissue and thus forcing the fluid to move within the tissue towards a lower-pressure location. If the pressure is applied too far from the fluid extraction location, this positive pressure gradient fades away into the surrounding skin and underlying tissues before it reaches the passage location with sufficient magnitude to achieve extraction of fluid from the skin. The size of the rim, both in terms of distance from the micro-needle location and skin contact area, thus plays a crucial role in the success of the sampling procedure.

Another important factor for the success of the sampling, especially but not only in the case in which the fluid to be collected is a pure interstitial fluid sample, is to separate and de-couple as much as possible the resulting force applied to the rim and to the sampling unit. In fact, if the same force is equally transferred to the sampling unit and to the rim, the micro-needles protruding from the body might penetrate more than necessary the compressed skin tissue and result in an increased risk of bleeding and thus blood contamination into the interstitial fluid sample. Another problem with skin tissue compression in correspondence of the micro-needle area is the increased fluidic resistance generated into the tissue, impeding or restricting fluid flow and potentially stopping the fluid extraction.

In the drawings, the sampling unit is not always explicitly shown, for illustration simplicity, but it is always attached or combined in a certain manner to the applicator to form a fully functional device in all embodiments.

FIG. 4c is a cross-sectional side view of the device, composed of cutaneous force applicator 310 and sampling unit 100. The rim portion 320 of the applicator, when a force is applied onto the applicator, exercises a positive mechanical pressure to the skin tissue as described above.

FIG. 4a is a top-view cross-sectional illustration of the sampling unit, where in this embodiment example one micro-needle 115 is placed in the center (perpendicularly to the view plane) and the storage unit contains a porous retaining material 130. During the sampling procedure the fluid fills the proximal end starting from the location where the passage abuts the retaining material and flowing towards the distal end. A colored dye may be present in the retaining material 130 to enhance visualization of the progression of the fluid front 150 into the retaining material 130. Thanks to the pre-defined geometry and characteristics of the retaining material, when the fluid reached the distal end, which can be visualized by the operator through a visualization window 142, the operator gets a confirmation that a specific volume has been collected and that the procedure is complete and shall be interrupted.

In a specific embodiment, used only as an example but not restricting the described device to only using these dimensions, some relevant sizes and inter-distances may include: a sampling unit with a circular orthogonal projection with diameter of 5 mm, a radial distance between sampling unit and rim of approximately 1 mm, a radial width (defined as difference between inner and outer radius) of the rim shaped as a ring of 0.75 mm.

FIG. 5a is a perspective view of a device 100, also referred to as a sampling unit 100, and a pressure generating means or cutaneous force applicator 200, which may be similarly configured as the devices 100 and pressure generating means 200 of the previous figures. Thus, the pressure generating means or cutaneous force applicator 200 may comprise a rim portion, such as a ring surrounding the sampling unit 100, forming part of a holder configured to releasably receive the sampling unit 100. This allows for the sampling unit 100 to be removed and the holder 200 to be reused. In another example, the connection between sampling unit 100 and pressure generating means 200 may not be releasable, making the entire device single use. The holder 200 may be attached to the skin 10, such as for example the skin of an arm of a human, by means of an attachment means 230. The attachment means 230 may be shaped as an armband that can be tied to the arm, such as an elastic or rigid band.

FIG. 5b is a perspective view of a device, also referred to as a sampling unit 300, and a pressure generating means or cutaneous force applicator 310, which may be similarly configured as the previously-described devices and pressure generating means. Thus, the pressure generating means may comprise a rim portion 320, such as a ring surrounding the sampling chip, forming part of a holder configured to be integrated, reliably or permanently, with the sampling unit. When the connection is releasable, this allows for the sampling device to be removed and the shooting device to be reused. The shooting may be pressed to the skin, such as for example the skin of an arm of a human, by an external individual, most likely a trained user or the patient itself.

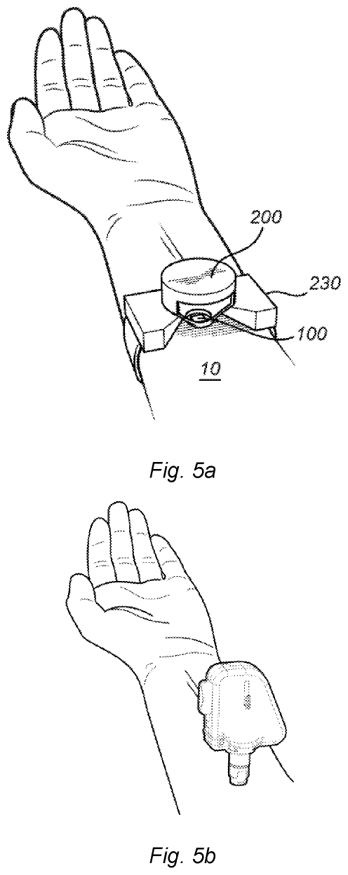

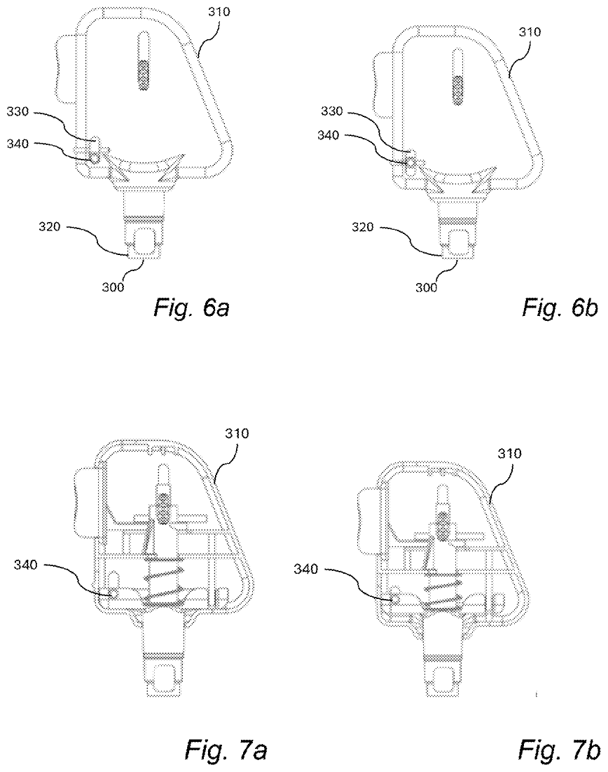

FIG. 6a is a side view of the device in its resting position, meaning that no force is applied and consequently there is no interaction or movement between the various components of the device. It is noticeable how the pressure indicator mark 340 is located at the lowest position of the slot 330 that allows it to slide vertically; this confirms that no force is applied to the device and therefore no pressure is transmitted to the skin surface; further description will be given during the explanation of FIG. 6b.

FIG. 6b is a side view of the device when a force is applied from the upper side by the user. Unlike the previous image, FIG. 6a, here the pressure indicator mark 340 is located halfway up the slot 330, corresponding to the same height as the ideal force signal.

The applied force may be in a range between 0.5 N and 20 N; preferably, the range may be between 4 N and 15 N; most preferably, the range may be around 10 N±2 N.

In a study where the respective dimensions of the rim, micro-needle and sampling unit were as described above, an exemplary ideal force was approximately 10 N, which is the amount of force needed to extract an ISF sample in a reasonable amount of time, on average 5 minutes, while avoiding side effects, excessive patient discomfort, or unwanted bleeding. This mark was positioned at that precise altitude because it considers the spring constant of a specific spring in relation to its compression distance. This is explained in more detail in FIG. 7b.

In a similar embodiment, the force indicator on the scale may comprise multiple indicators to signal an applied force within a range, rather than a specific value, where the range is the one suitable for sample extraction. The height of the slope allows the pressure indicator mark 340 to slide further upward, as it is possible to apply a greater force, and therefore pressure (almost double). This would benefit sampling time, which would decrease, but at the expense of discomfort for the patient. It is important for the pressure value to exceed a minimum threshold corresponding to the liquid retaining capability of the skin, and to avoid excessive pressure that could result in discomfort for the patient or other side effects such as bruising or, in the case of interstitial fluid extraction, undesired bleeding and thus blood contamination.

The pressure, applied by the cutaneous force applicator to the skin surface, may be in a range between 0.05 MPa and 2.5 MPa (megapascals); preferably the pressure may be between 0.25 MPa and 1.5 MPa; most preferably the pressure may be around 1 MPa±0.4 MPa.

FIG. 7a is a cross section of the device in its resting position, meaning that no force is applied and consequently there is no interaction or movement between the various components of the device.

It is noticeable how the pressure indicator mark is located at the lowest position of the slot that allows it to slide vertically; this confirms that no force is applied to the device and therefore no pressure is transmitted to the skin surface. Unlike FIG. 6a, where the device shell conceals its internal components, this image reveals the internal architecture. It therefore allows visualization of the components that make up the pressure indication system: half the shell with a carved slope, a spring, and a pressure component with its printed mark.

Although in this rest position there is no movement between the parts, it is noticeable how the spring is wedged between a rib of the horizontal shell and the pressure component; the latter is secured to the shell by both the vertical rib and the carved slope, where the pin with the printed pressure mark can potentially slide vertically;

FIG. 7b is a cross section of the device when a force applied from the upper side is therefore moving the pressure indicator vertically according to an embodiment. Unlike FIG. 6b, where the device shell conceals its internal components, this image reveals the same internal architecture of FIG. 7a. It therefore allows once again visualization of the components that make up the pressure indication system. Now that a force is applied, the interaction of the internal components can be observed. The force applied to the external shell from top to bottom compresses the spring (which is in contact with the horizontal rib and the pressure component). The user can apply a desired amount of force ranging from 0N to the mechanical target given by the slope carved into the shell where the pressure indicator can slide vertically. Only when it reaches the signal printed on the shell (see FIG. 6a) is the desired value of force applied, in this case this force was approximately 10N, and therefore generating an exerted pressure on the skin surface in the desired range for optimal extraction of ISF. As in FIG. 7a, the pressure component is secured to the shell by both the vertical rib and the carved slope, where the pin with the printed pressure mark can potentially slide vertically. It should be noted that, for blood sampling, the pressure commonly needed is slightly lower and does not need to be applied for as long of a time, since circulating blood possesses an intrinsic hydraulic pressure and, once a wound is created in a capillary, the only counter-effect to overcome to extract a blood sample from the skin is the elastic closure of the skin wound, and not the retaining of the fluid by the skin, nor the fluidic resistance for more fluid to flow through the skin matrix itself. In fact, most commonly, for blood extraction a sampling unit configuration similar to the one described in FIG. 13 would be employed, where the micro-needle is at least partially retracted out of the wound created. Therefore, a typical time for force application for blood sampling would be between 2 seconds and 1 minute, most typically between 5 and 20 seconds, and a typical range would be between the typical and the lower end of the ranges stated above for force and pressure, most typically a pressure between 0.1 and 0.5 MPa.

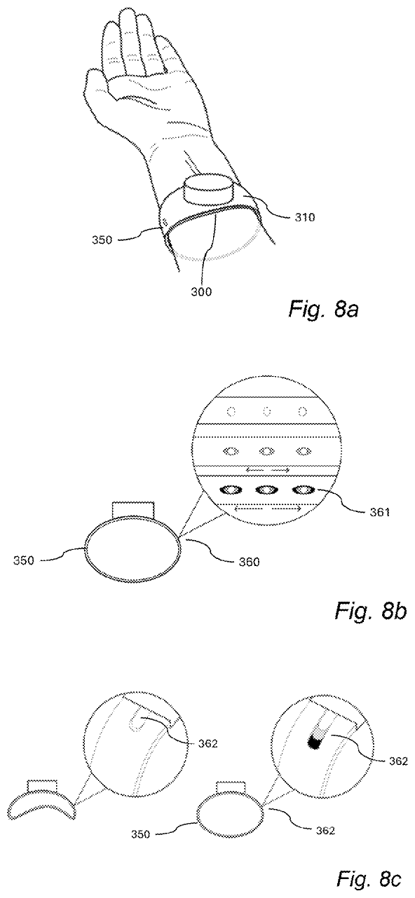

FIG. 8a is a perspective view of the device, composed of a sampling unit 300 and a cutaneous force applicator 310. The cutaneous force applicator is also a force/pressure generating means. The pressure generating means may comprise a rim portion 320, such as a ring surrounding the sampling unit. The ring may be pressed against the skin, such as for example the skin of an arm of a human, by an elastic band 350 responsible for securing the device to the body and applying a force onto the rim when placed on a forearm of a subject.

FIGS. 8b and 8c show two possible embodiments where a force gauge is integrated with the applicator in correspondence of the elastic band 350 and its elongation during use. In the embodiment of FIG. 8b the elastic band exposes a sign or a different color 361 through the stretched fibers or pores when the pulling force exceeds a pre-determined threshold amount. In the embodiment in FIG. 8c, a scale 362 is placed in relation to the elastic band. This scale may be color-coded, for example by providing three ranges where force is insufficient, in-range or excessive, or graded providing a numerical value, range, or equivalent indication to an operator.

In alternative embodiments based on elastic bands, instead of providing feedback to an operator via a scale or visual indication, a mechanism could click or lock in position when the force is surpassing a pre-determined threshold. The information about the locking might be realized via a sound emitted mechanically or directly lock the pull by the elastic band.

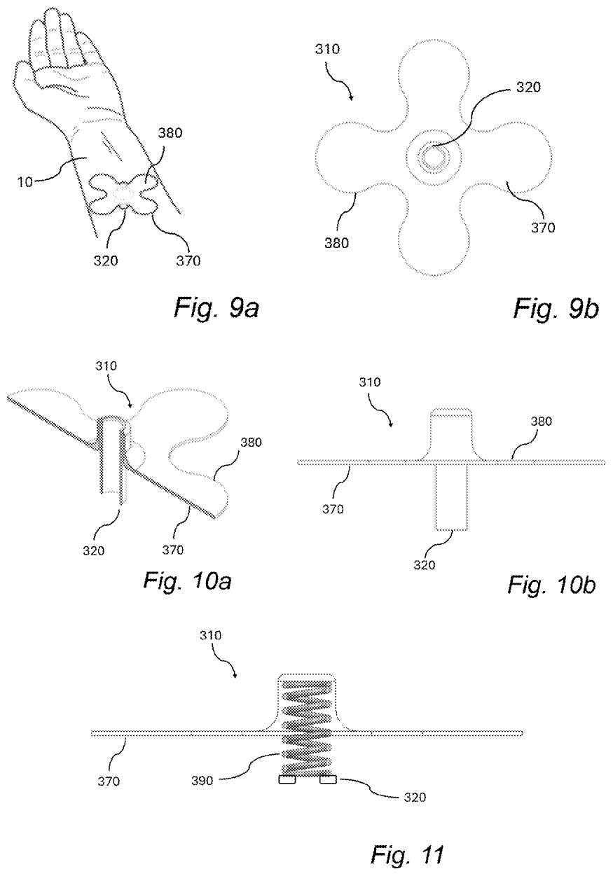

FIG. 9a is a perspective view of the cutaneous force applicator where the pressure generating means may comprise a rim portion 320, such as a ring surrounding the sampling unit. The ring may be pressed to the skin 10, such as for example the skin of an arm of a human, by an operator and then be kept in position by an adhesive material 370 which is positioned on a bottom surface of a portion of the applicator arranged around the central ring on a different plane, typically parallel. This surface 380, in a similar embodiment, might be slightly curved and/or shaped with fin-like geometries (such as in the enclosed drawings) to enable conforming to the skin surface which is not flat in most body locations, while preserving enough area to guarantee sufficient adhesive force to counteract the elastic forces provided as a result of the tissue compression and its elasticity.

FIG. 9b is a top view of the cutaneous force applicator 310 where the device is attached to the skin via an adhesive material 370. From this angle, it is noticeable the geometry of the fins 380 and therefore the contact surface with the skin where the adhesive layer 370 370 is applied. The shape is not random; rather, it allows for a compromise between optimized extension and flexibility of the fins themselves, which must adapt to the rounded surface of the human body, evenly distributing the tensile stress that would be created by sharper edges. Nonetheless, the function is not dependent on this specific geometry of the adhesive and can operate with simpler designs, such as for example a circular or rectangular perimeter.

FIG. 10a is a perspective view of the cross-section of the cutaneous force applicator already illustrated from a different perspective in FIGS. 9a-b, where the central cylindrical component is rigid and its bottom is acting as the rim portion 320 providing pressure onto the skin. In this embodiment the force/pressure amount is pre-defined by the indentation depth of the rim into the body tissue, possible thanks to the elastic properties of the skin, with respect to the plane where the adhesive 370 is attached to the skin, at least partially surrounding externally the rim 320.

FIG. 10b is a side view of the same cutaneous force applicator where the central cylindrical component is rigid and its bottom is acting as the rim portion 320 providing pressure onto the skin. By pressing the device onto the skin, the rim portion will indent into the tissue until the skin touches and attaches to the adhesive 370 around it, resulting in a net force applied to the skin by the rim 320 whose magnitude depends on the difference in height between the planes of the rim and the adhesive patch 370, also in relation to the elastic properties of the skin.

FIG. 11 is a side view of the cutaneous force applicator in an embodiment where the attachment means is the same as FIGS. 9A and 9B, but where the force/pressure is applied on the skin via the rim 320 by a spring-loaded mechanism, such as for example a metal coil spring 390, a curved plastic or steel leaf spring, a plastic bellow or flexural dome. The rim could be formed by the end of a coil spring itself or by any other material and rim geometry place in between the force-controlling mechanism and the skin. The force is controlled and defined by the spring compression. The advantage of this embodiment compared to that of FIGS. 9a and 9a is that it more easily accommodates body locations with less soft tissue present beneath the rim. In these configurations, illustrated in FIGS. 7a-b and 9a-b, it is important to control the distance between the rim 320 and the inner part of the adhesive surface 370, to avoid significant differences between body locations with softer or harder tissue beneath the skin, while allowing enough space for the skin to accommodate its needed elastic deformation.

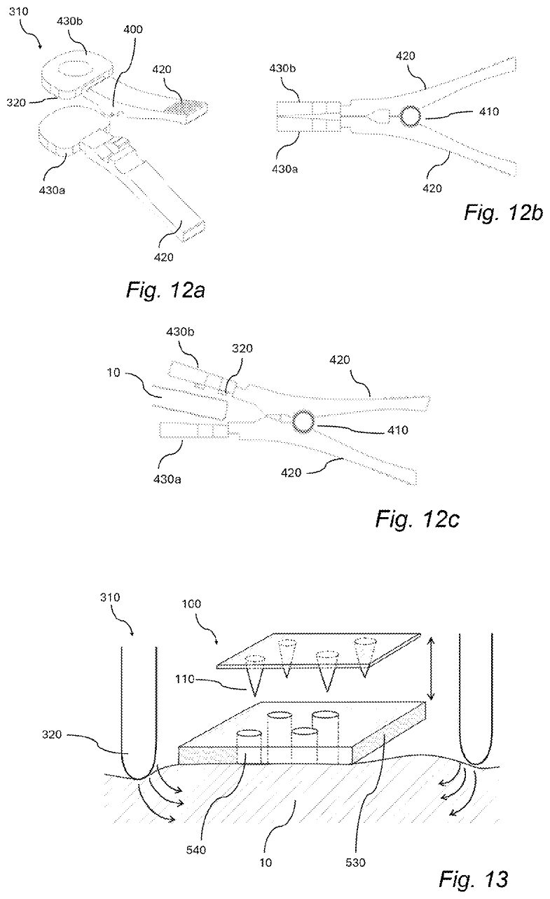

FIG. 12a is a perspective view, in the form of an exploded-perspective view, of the cutaneous force applicator 310 where the means to attach is a clothespin-like or clip-like mechanism 400 used to pinch a portion of skin and underlying tissue, and where the spring-based mechanism of the clip itself applies the desired force via the rim portion 320 onto the skin tissue. FIG. 12a highlights the geometry of the two elements that make up the clip in an exemplary embodiment. Between these two arm components 420 a spring-based mechanism is placed, for example a torsion spring 410, allowing the desired force to be applied (the spring is not visible in this drawing). From this drawing it is possible to appreciate a perforation in one of the plates, which represents the location where the remaining components, such as a a spring-loaded inserter and a sampling unit 300, interface the clip-like portion of the cutaneous force applicator 310.

FIG. 12b is a side view of the support device in the closed position, where between the two clip plates 430a and 430b a skin portion would be pinched. The clip-like mechanism is used to pinch a skin portion and thereby both fixating the device to the body and at the same time applying a defined force/pressure to the tissue underneath, via the rim present on at least one of the two clip plates. What can be appreciated in the drawing is its resting position. To open it, an external force must be applied along the levers 420 that allow the clamps to open, pinch a portion of tissue, and then close thanks to the spring force and its willingness to return to the initial closed position. In a similar embodiment the mechanism might be implemented with a different spring configuration to obtain the same effect. In a possible embodiment drawn in FIG. 12b it is also possible to notice that the surface of the two clamps is not perfectly horizontal but is angled to account for the opening angle at which the clip faces are positioned while clamping the skin tissue; it is important for the surface of the clip faces to be parallel to the surface of the clamped skin, to allow subsequent firing of the sampling chip along the vertical axis, perpendicularly, as well as to apply a homogeneous force (and thus compression) to the pinched tissue.

FIG. 12c is a side view of the same applicator in the open position, where between the plates 430a and 430b a skin portion is present. Upon closing the clip mechanism, the skin is pinched in position, and the pressure is applied to the skin tissue via the rim on the bottom side of the top plate of the clip.

FIG. 13 is a perspective cross-sectional view of a device, where in this example the cutaneous force applicator 310 can be any of the embodiments described above, while the sampling unit 100 is composed as follows. The at least one micro-needle 110 is not necessarily featured with a passage, such as a solid micro-needle, and is first applied to the skin 510 to generate a pore or wound in the skin and then retracted before the extraction of the sample is started. During the procedure, the storage unit 530 is placed in proximity of the pores generated by the penetration to collect the fluid sample as soon as it starts flowing, upon application of a pressure to the skin tissue by the rim 320. In this example, a retaining material 530 is thus placed in proximity to the skin and attached to the applicator 310 rather than the body of the sampling unit 100 and features holes in correspondence of the path of the micro-needles 110 during insertion and retraction, not to interfere with their ability to travel towards the skin 10 and penetrate it.

This configuration is of particular interest for blood sampling, since the retraction of the micro-needles facilitates the blood flow out of the skin, but is nonetheless applicable to the sampling of ISF as well, depending on the size and geometry of the micro-needles (or blades, or any other wound-generating element) and thus the resulting wound or pore generated into the skin upon penetration.

It is intended that all the different embodiments presented, in terms of designs and mechanisms examples of the various sampling units, the applicators, and their described sub-components can be combined and exchanged as it is obvious to the person skilled in the art.

### Experimental Results

A device according to an embodiment of the present disclosure was evaluated in a study, which is recited in the following to further exemplify embodiments and possible use of the invention. The device was composed of two main parts: (i) the sampling device 100 similar to the ones described above, and (ii) an inserter, or holder 200, comprising a pressure generating means, or pressure ring, according to the above embodiments. The inserter was used for inserting the micro-needle in the skin and for the generation of a mechanical overpressure facilitating overcoming the physiological ISF retention by the skin to guide the ISF into the micro-needle lumen.

Volume-metering was provided by the geometry of the retaining material, in this example paper, and by a fill level indicator. In the present example the fill level indicator comprised coloured dye present in the paper itself. During sampling the liquid progressed within the paper matrix transporting the soluble coloured dye. Once the liquid reached the visualization window in the body, the device was removed from the skin.

The device, also referred to as a microfluidic chip, was designed to collect about 1 μl of ISF. The reliability was characterised in vitro. In most cases, sampling was completed within 5 minutes, with a variation between 3 and 8 minutes. The total number of samplings was in this study 15. The micro-needle insertion was reported as painless by all volunteers, in this case humans. The samples were analysed using liquid chromatography-tandem mass spectrometry (LC-MS/MS), with a similar protocol to the one developed for dried blood spots (DBS) analysis.

The metering accuracy was evaluated by pumping an artificial ISF solution (phosphate buffered saline solution, glucose, bovine serum albumin, dye) into the microneedle lumen at defined flow rates, which were varied between the different runs. At the time in which the liquid crossed the marking of the visualization window, the flow was stopped, and the total amount dispensed by the toll was recorded. For these tests, a syringe pump was used. The experiments were video recorded using a portable microscope camera.

To verify the possibility to measure relevant physiological concentration of analytes in small sample volumes by means of embodiments of the present invention, the concentration of caffeine in 1 μl spiked ISF surrogate was measured, as an example of a target molecule. The different concentrations in the various samples were successfully measured using LC-MS/MS, with protocols previously developed for analysis in whole blood from DBS paper.

For the presented measurements, 1 μl aliquots of dyed artificial ISF with the addition of caffeine at concentrations varying from 0.1 microgram per millilitre (μg/ml) to 9 μg/ml were pipetted into different paper pieces, in duplicates. Samples were then let dry into the paper matrix and stored. The dried samples were then evaluated and characterised using LC-MS/MS. Detection of caffeine was also confirmed in real ISF samples extracted from a human volunteer. Finally, to evaluate the penetration depth and the effect of microneedle penetration into the skin, an optical coherence tool was used.

The samples were collected on healthy human volunteers. The chosen sampling location was in the inner forearm, having a skin with layer thicknesses allowing access to the dermal region using 1 mm long microneedles. The sampling continued until the indicator was visualised by the operator unless the volunteer decided to interrupt the procedure earlier.

The volume-metering reliability of the used device was assessed by pumping an ISF surrogate solution into the device at known flow rates until it reached the visualisation window. The minimum and maximum flow rates used were 0.13 μl/min and 0.34 μl/min, respectively. These values correspond to the fastest and slowest sampling rates observed during in-vivo experiments. By imposing a flow rate of 0.13 μl/min, the indicator location was reached after dispensing 1.06 μl, while with 0.34 μl/min, 1.13 μl were dispensed. Therefore, the difference between the minimum and maximum flow rates was within +/−3.5%, with an average collected volume of about 1.1 μl.

The in-vivo sampling was performed by applying the device to the forearm. In this study, a spring-loaded inserter and a pressuring ring were used to achieve insertion and apply a radial force of about 10 N to lead dermal ISF to the micro-needle opening, respectively. The process was stopped when the liquid front reached the indicator location, in this example the edge of the visualisation window illustrated in FIG. 4a. The sampling time varied between 3 and 8 minutes (n=15) between different tests and individuals, with an average of 5 minutes.

The insertion speed and the micro-needle geometry may affect the skin penetration process due to the elastic properties of the skin. An OCT image of the needle inserted in the skin was acquired. The skin bending around the needle was approximately 150 μm. The needle opening, starting about 250 μm below the device base, was thus completely inserted inside the skin. One hour after the removal of the needle the pricking location could not be discerned using the OCT tool. The micro-needle insertion was reported as painless by all the individuals. Initially, signs of the skin pricking and the ring pressure were visible at and around the sampling location. The skin recovered within five hours. No permanent mark was visible days after the experiments.

The results of the experiments recited herein indicate that the embodiments of the present disclosure provide an efficient and minimally invasive technique for sampling ISF from mammal subjects. Compared to prior art techniques, the present device may be simpler, more compact and potentially more cost-effective due to the choice of materials, the fabrication techniques involved, and the lack of complex actuators. Further, the device may be disposable and possible to use with a reusable inserter or holder for facilitating extraction of the ISF sample from the skin.

The inventive concept has mainly been described above with reference to a few embodiments and examples. However, as is readily appreciated by a person skilled in the art, other embodiments than the ones disclosed above are equally possible within the scope of the inventive concept as defined by the appended claims.

1. A device for extracting a fluid sample from a skin of a subject, comprising:
at least one micro-needle comprising a tip portion, an end portion, and a passage, wherein the tip portion is configured to be inserted into the skin of the subject, and wherein the passage is configured to transport the fluid sample, extracted from the skin by the at least one micro-needle, away from the tip portion;
a body configured to support the at least one micro-needle;
a storage unit arranged in the body, the storage unit fluidically connected to the passage and configured to collect and temporarily store the fluid sample transported by the passage; and
a cutaneous force applicator configured to apply a repeatable, controlled force to the skin tissue around the micro-needle to generate a controlled pressure on the skin tissue, the pressure configured to extract a fluid sample from the skin.

2. The device according to claim 1 where the fluid sample is one of an interstitial fluid, or a capillary blood fluid.

3. The device according to claim 1, wherein the cutaneous force applicator comprises a rim portion configured to abut the skin and to at least partially encircle the at least one micro-needle when said at least one micro-needle is inserted in the skin, wherein the rim portion is further configured to exert a pressure on portions of the skin adjacent to the at least one micro-needle to facilitate extraction of the fluid sample from the skin.

4. The device according to claim 1 wherein the controlled pressure is configured to provide the same amount of tissue compression and equivalent extraction efficacy.

5. The device according to claim 1 wherein the pressure applied compresses the skin tissue to induce transport of the fluid sample towards the micro-needle tip portion and the micro-needle passage.

6. The device according to claim 1 wherein the pressure onto the skin is applied manually by a human operator onto the device throughout the fluid extraction procedure.

7. The device according to claim 6 wherein the cutaneous force applicator is designed to provide a feedback to the human operator about the amount of pressure applied and/or about being in a specific range of the desired pressure.

8. The device according to claim 7 wherein the cutaneous force applicator comprises a visual indicator having a movable mechanical part, wherein the visual indicator is configured to provide the feedback about being in a specific range of the desired pressure.

9. The device according to claim 8 wherein the movable mechanical part is connected to a spring mechanism being pulled or compressed with a force configured to apply the desired pressure to the skin tissue.

10. The device according to claim 1 wherein, after the device is initially set up, the controlled and reproducible pressure, is applied by the device to the skin tissue without further manual force from or control of the pressure by a human operator throughout the entire extraction of the fluid sample.

11. The device according to claim 10, said device further comprising an elastic band configured to secure the device to the skin and to apply a repeatable, reproducible, controlled pressure to the skin tissue via the rim portion of the cutaneous force applicator.

12. The device according to claim 11 wherein the pressure is measured by a pressure gauge on the elastic band wrapped around a location on the skin of the subject where the extraction procedure occurs.

13. The device according to claim 12 wherein the pressure gauge is configured to expose a color change or a sign appearance in or through the elastic band due to a calibrated stretching of fibers and/or components of the elastic band.

14. The device according to claim 11 wherein, when the pressure applied onto the device towards the rim portion surpasses a defined threshold, a mechanism clicks and/or locks in position providing the feedback to the operator about readiness to start the extraction.

15. The device according to claim 10, wherein the device is attached to the skin via an adhesive material.

16. The device according to claim 14 wherein the pressure is applied on the skin via the rim portion by a spring-based mechanism, such as for example a metal coil spring, a curved plastic or steel leaf spring, a plastic bellow or flexural dome.

17. The device according to claim 10, said device further comprising a clip, the clip comprising a spring-based mechanism arranged between two clip arms, the clip configured to pinch a portion of skin and tissue between the two clip arms.

18. The device according to claim 17 wherein the spring-based mechanism of the clip applies the desired force via the rim portion onto the skin tissue.

19. The device according to claim 1 wherein said applied force is in a range between 0.5 N and 20 N, more typically between 4 N and 15 N, most typically around 10 N±2 N.

20. The device according to claim 1 wherein said pressure, applied by the cutaneous force applicator to the skin surface, is in a range between 0.05 MPa and 2.5 MPa (megapascals), more typically between 0.25 MPa and 1.5 MPa, most typically around 1 MPa±0.4 MPa.
